(19) United States
(12) Patent Application Publication
(10) Pub. No.: US 2013/0096423 A1
(43) Pub. Date: Apr. 18, 2013

(54) ENDOSCOPE SYSTEM

(71) Applicant: OLYMPUS MEDICAL SYSTEMS CORP., Tokyo (JP)

(72) Inventors: Tatsuro YAMAMOTO, Hachioji-shi (JP); Jun HASEGAWA, Hino-shi (JP)

(73) Assignee: OLYMPUS MEDICAL SYSTEMS CORP., Tokyo (JP)

(21) Appl. No.: 13/626,668

(22) Filed: Sep. 25, 2012

Related U.S. Application Data
(63) Continuation of application No. PCT/JP2012/054089, filed on Feb. 21, 2012.

(30) Foreign Application Priority Data
Mar. 30, 2011 (JP) 2011-075283

(51) Int. Cl. A61B 1/01; A61B 1/05; A61B 6/00; A61B 1/008; A61B 1/00; A61B 6/02

(52) U.S. Cl. CPC A61B 1/01; A61B 1/00043; A61B 1/00193; A61B 6/02; A61B 6/5217; A61B 1/008; A61B 1/05
USPC 600/424; 600/103

(57) ABSTRACT

An endoscopic system includes an insertion portion inserted into a tubular body, a ranging mechanism, an insertion path calculation unit and a presentation unit. The insertion portion includes a distal end and a bending portion defining a driving face. The ranging mechanism acquires distance information on the driving face between an inner wall of the tubular body on a far side and the distal end of the insertion portion while the distal end is placed on a near side in the tubular body. The insertion path calculation unit calculates an insertion path for the distal end of the insertion portion extending from the near side on which the distal end is placed, to the far side, based on the distance information. The presentation unit presents the insertion path for the distal end of the insertion portion extending from the near side to the far side.

### CROSS-REFERENCE TO RELATED APPLICATIONS

This is a Continuation Application of PCT Application No. PCT/JP2012/054089, filed Feb. 21, 2012, which was published under PCT Article 21(2) in Japanese.

This application is based upon and claims the benefit of priority from the prior Japanese Patent Application No. 2011-075283, filed Mar. 30, 2011, the entire contents of which are incorporated herein by reference.

### BACKGROUND OF THE INVENTION

1. Field of the Invention

The present invention relates to an endoscopic system which can support the insertion of the insertion portion of an endoscope from the near side in a tubular body to the far side.

2. Description of the Related Art

For example, International Publication No. 2010/046802 Pamphlet discloses a system which obtains a shape of the bronchus in advance by using a CT scanner, then estimates the inserted state of the insertion portion of an endoscope when it is actually inserted into the bronchus, and can display an image depicting how the insertion portion is inserted into the bronchus.

Assume that the system disclosed in the International Publication No. 2010/046802 Pamphlet is used for a tubular body, such as the large intestine, which is not fixed in the body cavity and freely moves while freely deforming. In this case, even if the shape of the tubular body is measured in advance by a CT scanner or the like, the shape of the tubular body momentarily changes as the insertion portion of the endoscope is inserted. For this reason, when supporting the insertion of the insertion portion by, for example, allowing to comprehend the shape of a tubular body at the present moment and the direction in which the insertion portion is to be moved, by using the system disclosed in patent literature 1, it is necessary to use a CT scanner while the insertion portion of the endoscope is inserted. However, the CT scanner is very large medical equipment, and hence it is difficult to scan a freely moving tubular body such as the large intestine many times.

### BRIEF SUMMARY OF THE INVENTION

An endoscopic system according to the invention includes: an elongated insertion portion which is configured to be inserted into a tubular body and which includes, at a distal end portion, a bending portion which configured to freely bend; a position/posture detection unit which is configured to detect a position and posture of the distal end portion as position/posture information; an operation position/posture calculation unit which is configured to calculate, as driving face information, a position and posture of a driving face on which the bending portion bends, based on the position/posture information; a peripheral information detection unit which is configured to detect a bent crooked region of the tubular body existing on the driving face as peripheral information based on the driving face information; a positional relationship calculation unit which is configured to calculate a positional relationship between the bending portion and the bent crooked region as positional relationship information based on the position/posture information, the driving face information, and the peripheral information; and a presentation unit which is configured to present the positional relationship based on the positional relationship information.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing an endoscopic system according to a first embodiment.

FIG. 2 is a schematic longitudinal sectional view of the bending portion of the insertion portion of the endoscopic system according to the first embodiment.

FIG. 3 is a schematic block diagram showing the endoscopic system according to the first embodiment.

FIG. 4A is a schematic view showing a state in which an observation image is obtained by using the observation optical system of the endoscope of the endoscopic system according to the first embodiment.

FIG. 4B is a schematic view showing the observation image shown in FIG. 4A.

FIG. 4C is a schematic view showing distance information of the inner wall of a tubular body relative to the distal end face of the distal end hard portion of the insertion portion of the endoscope at points a, . . . , k on the driving face in the U and D directions of the bending portion in FIG. 4B.

FIG. 5 is a schematic flowchart showing the operation of supporting the insertion of the insertion portion into a tubular body by using the endoscopic system according to the first embodiment.

FIG. 6A is a schematic view showing a closed state on the far side on the driving face F1 shown in FIG. 4A and distance information of the inner wall of a tubular body relative to the distal end face of the distal end hard portion on the driving face in the U and D directions of the bending portion of the insertion portion of the endoscope by using the endoscopic system according to the first embodiment.

FIG. 6B is a schematic view showing a state in which an insertion path exists on the far side on the driving face F1 shown in FIG. 4A and distance information of the inner wall of a tubular body relative to the distal end face of the distal end hard portion on the driving face in the U and D directions of the bending portion of the insertion portion of the endoscope by using the endoscopic system according to the first embodiment.

FIG. 6C is a schematic view simplifying the illustration shown in FIG. 6B on the driving face F1 shown in FIG. 4A and showing a state in which an arrow is added to a distant portion of an insertion path and distance information of the inner wall of a tubular body relative to the distal end face of the distal end hard portion on the driving face in the U and D directions of the bending portion of the insertion portion of the endoscope by using the endoscopic system according to the first embodiment.

FIG. 7A is a schematic view showing a closed state on the far side, distance information of the inner wall of a tubular body relative to the distal end face of the distal end hard portion on the driving face in the U and D directions of the bending portion of the insertion portion of the endoscope by using the endoscopic system according to the first embodiment, and an example of a method of determining the existence of an insertion path and calculating the insertion path.

FIG. 7B is a schematic view showing a state in which an insertion path exists on the far side, distance information of the inner wall of a tubular body relative to the distal end face of the distal end hard portion on the driving face in the U and D directions of the bending portion of the insertion portion of the endoscope by using the endoscopic system according to the first embodiment, and an example of a method of determining the existence of an insertion path and calculating the insertion path.

FIG. 8 is a schematic view showing distance information of the inner wall of a tubular body relative to the distal end face of the distal end hard portion on the driving face in the U and D directions of the bending portion of the insertion portion of the endoscope by using the endoscopic system according to the first embodiment and an example of a method of determining the existence of an insertion path.

FIG. 9 is a schematic block diagram showing an endoscopic system according to a second embodiment.

FIG. 10 is a schematic view showing the arrangement of part of the endoscopic system according to the second embodiment.

FIG. 11 is a schematic view showing a method for obtaining a state in which the distal end portion of the insertion portion of the endoscope is superimposed on a tubular body by using the endoscopic system according to the second embodiment, with X-ray tomographic images and a detector.

FIG. 12 is a schematic block diagram showing an endoscopic system according to a third embodiment.

FIG. 13 is a schematic view showing the bending driving mechanism of the endoscope of the endoscopic system according to the third embodiment.

### DETAILED DESCRIPTION OF THE INVENTION

The embodiments of the present invention will be described below with reference to the accompanying drawing.

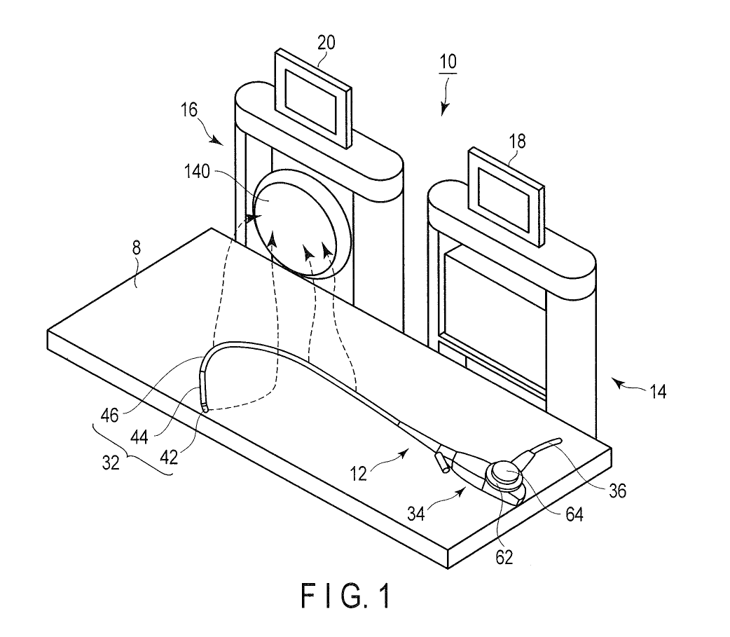

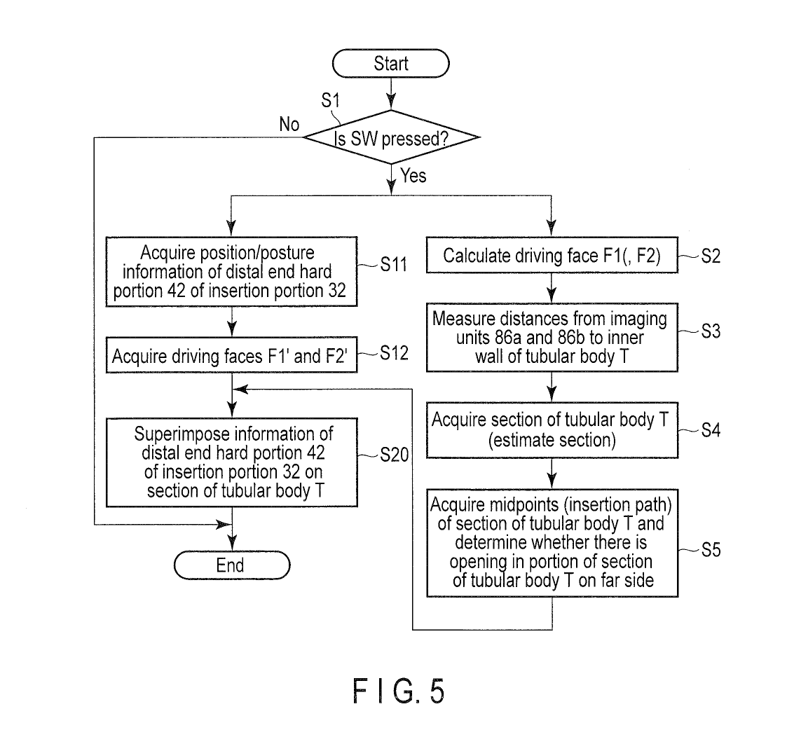

The first embodiment will be described with reference to FIGS. 1 to 6C.

As shown in FIG. 1, an endoscopic system (an insertion support apparatus for the insertion portion of an endoscope) 10 according to this embodiment includes an endoscope 12, a video processor 14, a detector (position/posture detection unit) 16, and monitors (presentation units, image display units) 18 and 20. The video processor 14 and the detector 16 are arranged near a bed 8. For example, one monitor 18 is disposed on the processor 14, and the other monitor 20 is disposed on the detector 16. One monitor 18 displays, for example, an observation image obtained by an observation optical system 74 (to be described later). The other monitor 20 displays, for example, the shape of an insertion portion 32 (to be described later) which is detected by the detector 16. The monitors 18 and 20 are connected to each other via the video processor 14 and the detector 16 and can display various kinds of information. That is, for example, both an observation image and the shape of the insertion portion 32 can be displayed on one monitor 18.

The endoscope 12 includes the elongated insertion portion 32 to be inserted into a tubular body such as a body cavity, an operation portion 34 which is disposed on the proximal end portion of the insertion portion 32 and is held by the user, and a universal cable 36 extending from the operation portion 34. The universal cable 36 detachably connects the endoscope 12 to the video processor 14 and the detector 16, respectively. Note that the video processor 14 and the detector 16 are connected to each other such that they can output and input data to and from each other.

The insertion portion 32 includes a distal end hard portion (the distal end portion of the insertion portion 32) 42, a bending portion 44 (the distal end portion of the insertion portion 32), and a flexible tube portion 46, which are sequentially arranged from the distal side to the proximal side. Note that the distal end portion of the insertion portion 32 includes the distal end hard portion 42 and the bending portion 44.

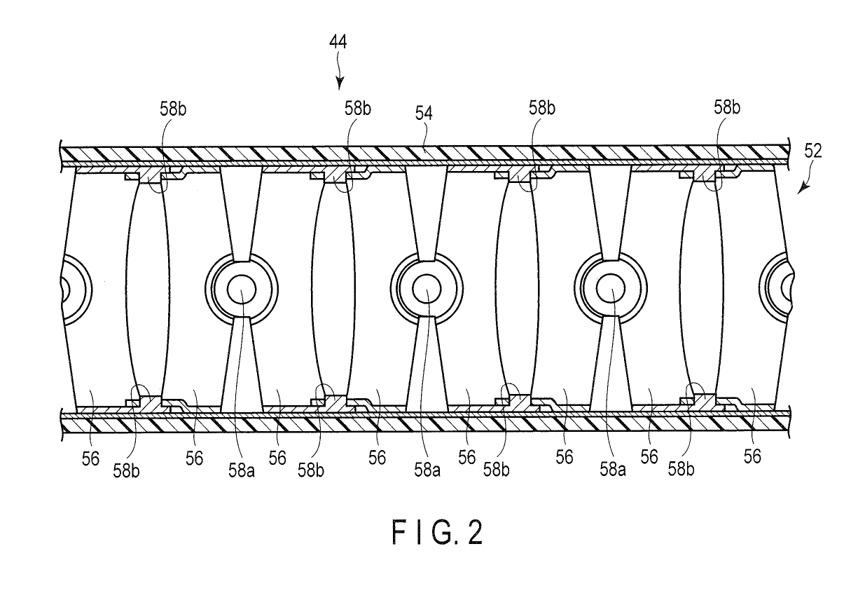

As shown in FIG. 2, the bending portion 44 includes a bending tube 52 and a outer tube 54 disposed outside the bending tube 52. The bending tube 52 has a plurality of bending pieces 56 coupled to each other through pivot shafts 58a and 58b. The first pivot shafts 58a of the bending tube 52 extend horizontally to allow the bending portion 44 to bend vertically. The second pivot shafts 58b extend vertically to allow the bending portion 44 to bend horizontally.

As shown in FIG. 1, the operation portion 34 includes angle knobs 62 and 64. Angle wires (not shown) are disposed between the bending piece 56 at the distal end of the bending tube 52 and the angle knobs 62 and 64 to allow to bend the bending portion 44 in the U and D directions by actuating the one angle knob 62 and to bend the bending portion 44 in the R and L directions by actuating the other angle knob 64.

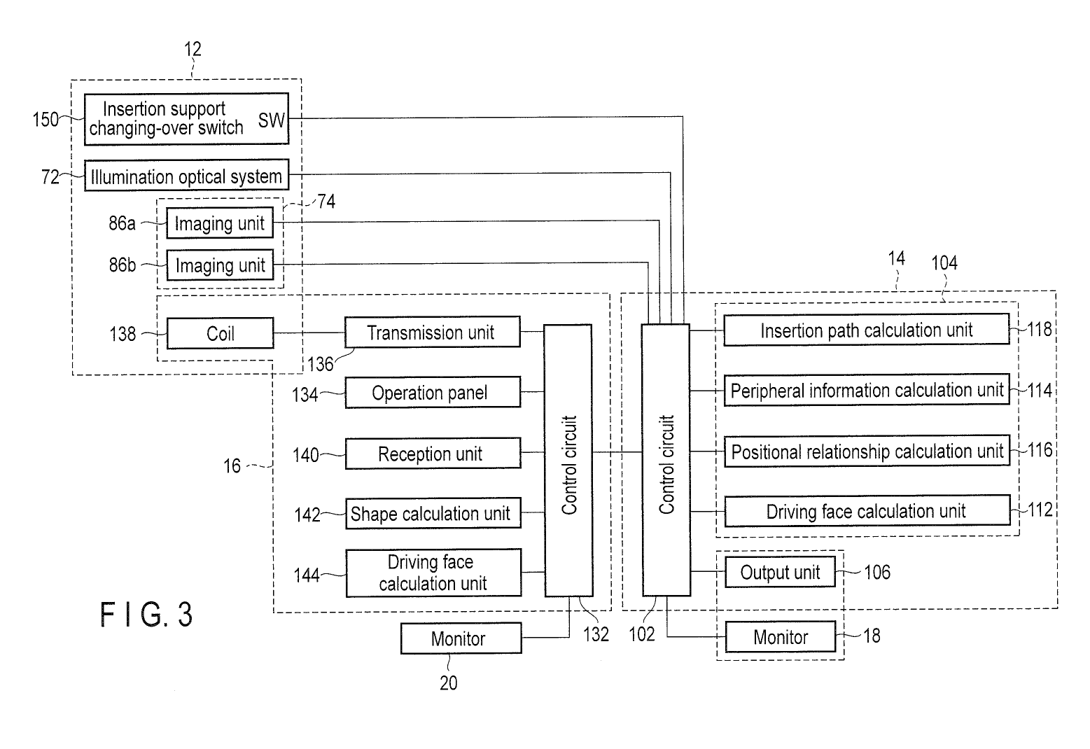

As shown in FIG. 3, an illumination optical system 72 and the observation optical system 74 are disposed in, for example, the insertion portion 32 and the operation portion 34 of the endoscope 12.

As the illumination optical system 72, for example, it is possible to use various types of light sources such as an LED and an incandescent lamp. It is possible to illuminate an object facing the distal end face of the distal end hard portion 42 by making illumination light emerge from the illumination lens disposed on the distal end of the distal end hard portion 42.

Note that using a compact light source allows it to be disposed at the distal end hard portion 42. In this case, the illumination optical system 72 is disposed at only the insertion portion 32.

The observation optical system 74 includes two objective lenses (not shown) and two imaging units 86a and 86b to implement stereo imaging (3D imaging). Image sensors such as CCDs or CMOSs of the imaging units 86a and 86b are preferably disposed inside the distal end hard portion 42 so as to be parallel with the distal end face of the distal end hard portion 42, with their vertical and horizontal directions being positioned in the same directions as bending directions. The embodiment will be described below on the assumption that the positions of the imaging units 86a and 86b are symmetrical about the central axis of the insertion portion 32 (in particular, horizontally symmetrical). For this reason, the vertical direction of the images captured by the image sensors of the imaging units 86a and 86b, i.e., the images displayed on the monitor 18 via the video processor 14 matches the vertical direction (U and D directions) of the bending portion 44, and the horizontal direction of the images matches the horizontal direction (R and L directions) of the bending portion 44.

When, for example, the pivot shafts 58a of the bending pieces 56 of the bending portion 44 shown in FIG. 2 are located in the horizontal direction, a driving face (bending face) F1 in the vertical direction (U and D directions) of the bending portion 44 is made to correspond to the vertical direction of the image sensors of the imaging units 86a and 86b. Likewise, when, for example, the pivot shafts 58b of the bending piece 56 are located in the vertical direction, a driving face (bending face) F2 in the horizontal direction (R and L directions) of the bending portion 44 is made to correspond to the horizontal direction of the image sensors of the imaging units 86a and 86b. That is, the driving face F1 is defined when the bending portion 44 bends in the U and D directions, and the driving face F2 is defined when the bending portion 44 bends in the R and L directions. For this reason, the user of the endoscope 12 can easily comprehend the bending faces (faces formed when the bending portion 44 bends) F1 and F2 of the bending portion 44 by only seeing the monitor 18.

The video processor 14 includes a control circuit 102, a operation part (calculation unit) 104, and an output unit 106. The output unit 106 is used to output various kinds of signals to various devices such as an automatic bending driving device 26 to be described in the third embodiment described later. The operation part 104 includes a driving face calculation unit 112, a peripheral information calculation unit (image processing unit) 114, a positional relationship calculation unit 116, and an insertion path calculation unit (bending direction calculation unit of the tubular body T) 118.

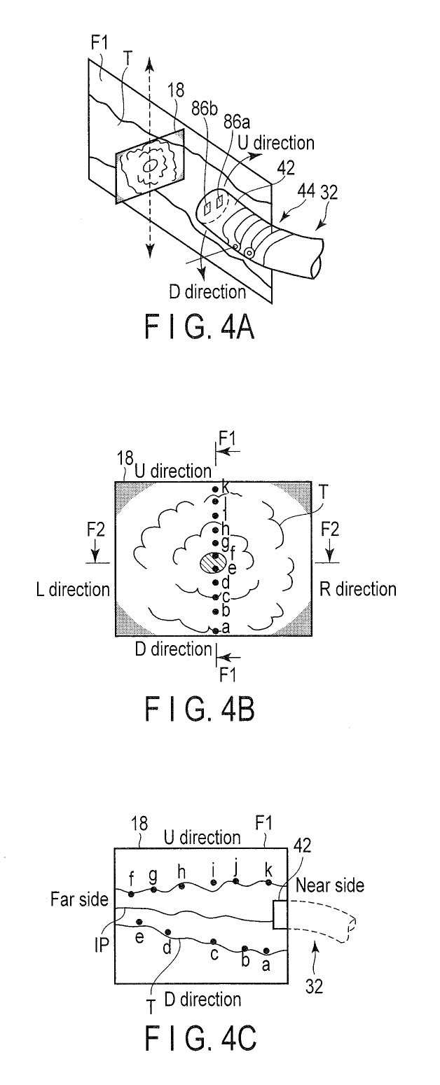

As shown in FIG. 4A, the driving face calculation unit 112 of the video processor 14 calculates the driving faces (bending faces) F1 and F2 of the bending portion 44 based on the image data information (peripheral information) obtained by the imaging units 86a and 86b. As shown in FIG. 4B, the monitor 18 can display the position of the bending face F1. The bending portion 44 can bend in the U and D directions and in the R and L directions, and hence the driving face calculation unit 112 can define the driving face F1 in the U and D directions and the driving face F2 in the R and L directions. Assume that in this embodiment, the imaging units 86a and 86b are located at positions in the middle in the vertical direction and laterally symmetrical with respect to the central axis of the insertion portion 32. For this reason, in the monitor 18, the driving face F1 is located in the middle in the horizontal direction, and the driving face F2 is located in the middle in the vertical direction.

The peripheral information calculation unit 114 of the video processor 14 calculates the distances between the image sensors of the imaging units 86a and 86b and the inner wall surface of a tubular body T at positions on the driving face F1. That is, the imaging units 86a and 86b and the peripheral information calculation unit 114 constitute a distance measuring mechanism for acquiring the distances between the image sensors of the imaging units 86a and 86b and the inner wall surface of the tubular body T at positions on the driving face F1. Note that the peripheral information calculation unit 114 can calculate not only the distances between the image sensors of the imaging units 86a and 86b and the inner wall surface of the tubular body T at positions on the driving face F1 but also the distances between the image sensors of the imaging units 86a and 86b and the wall surface of the tubular body T at positions falling outside the driving face F1.

In addition, the imaging units 86a and 86b and the peripheral information calculation unit 114 acquire the distances between the image sensors of the imaging units 86a and 86b and the wall surface of the tubular body T at positions on the driving face F1 and also acquire a peripheral observation image including the driving face F1, thus constituting a peripheral information detection unit.

The positional relationship calculation unit 116 matches coordinate systems based on the position. information and posture information (position/posture information) to be described later obtained by the detector 16 and the image data information (peripheral information) obtained by the observation optical system 74.

The insertion path calculation unit 118 calculates an insertion path IP along which the distal end hard portion 42 of the insertion portion 32 is inserted from the near side where it is placed to the far side in the tubular body T.

The endoscope 12 according to this embodiment includes two objective lenses and the two imaging units 86a and 86b. This makes it possible to measure spatial characteristics (distances) of an object by triangulation using the two image data obtained by imaging the object from two viewpoints. That is, the endoscopic system 10 can measure the distance to a given position on the object by image processing (by the peripheral information calculation unit 114) using a stereo matching method.

In this case, the stereo matching method is a technique of using the images captured by the two imaging units (cameras) 86a and 86b, and performing the image matching processing of searching for corresponding points between the respective points in the image captured by one imaging unit 86a and the respective points in the image captured by the other imaging unit 86b, thereby obtaining the three-dimensional position of each point in each image by triangulation, and calculating the distances.

The peripheral information calculation unit 114 vertically matches the middle region of the image displayed on the monitor 18 in FIG. 4B in the horizontal direction. That is, the peripheral information calculation unit 114 measures the distances from the imaging units 86a and 86b to the inner wall of the tubular body T on the driving face F1 in the U and D directions of the bending portion 44 at proper intervals. The distances from the imaging units 86a and 86b to the inner wall of the tubular body T can be expressed as shown in FIG. 4C. That is, it is possible to obtain a longitudinal section of the tubular body T on the driving face F1. Referring to FIG. 4C, since the driving faces F1 and F2 are defined by the imaging units 86a and 86b of the observation optical system 74, the U and D directions are automatically defined. In addition, the near side and the far side are automatically defined by the distal end face of the distal end hard portion 42.

In this manner, the endoscopic system 10 can obtain the distances from the distal end face of the distal end hard portion 42 to the wall surface of a tubular body on an image by using the principle of triangulation as well as obtaining an image of the inner wall of the tubular body T by stereo imaging. Therefore, collecting pieces of distance information with respect to the wall surface on the image can obtain a schematic shape of a longitudinal section of the tubular body T, as shown in FIG. 4C.

The detector (position/posture detection unit) 16 shown in FIG. 1 is used to measure the position and posture of the distal end portion of the insertion portion 32 of the endoscope 12, particularly the distal end hard portion 42. For example, a known endoscope insertion shape observation device (endoscope position detecting unit) (to be referred to as a UPD device hereinafter) can be used.

Although the following will exemplify the embodiment using the UPD device as the detector 16, it is possible to use various types of detectors such as a device designed to detect the position and posture of the distal end hard portion 42 of the insertion portion 32 by using a known FBG (Fiber Bragg Grating) sensor.

As shown in FIG. 3, the detector 16 includes a control circuit 132, an operation panel 134, a transmission unit 136, a plurality of magnetic coils 138, a reception unit 140, a shape calculation unit 142, and a driving face calculation unit (operation position/posture calculation unit) 144. Note that the detector 16 is to be used to detect only a shape, the detector may include only the control circuit 132, the operation panel 134, the transmission unit 136, the plurality of magnetic coils 138, and the reception unit 140.

The operation panel 134, the transmission unit 136, the reception unit 140, the shape calculation unit 142, and the driving face calculation unit 144 are connected to the control circuit 132. The plurality of magnetic coils 138 are incorporated in the insertion portion 32 at proper intervals and connected to the transmission unit 136. The magnetic coils 138 are incorporated especially in the portion between the distal end hard portion 42 and the flexible tube portion 46 at proper intervals. Note that the operation panel 134 is used to make various settings for the detector 16. The monitor 20 can display operation contents at the time of operation of the operation panel 134 and the currently estimated shape of the insertion portion 32 using the detector 16.

As shown in FIG. 1, the detector 16 generates a weak magnetic field by driving the plurality of magnetic coils 138 incorporated in the insertion portion 32 at different frequencies from the transmission unit 136, receives the weak magnetic field via the reception unit 140, and calculates the reception data using the calculation unit 142, thereby obtaining the information of the positions and postures (position/posture information) of the distal end hard portion 42 and bending portion 44 of the insertion portion 32 including the distal end hard portion 42. Note that connecting the calculated positional coordinates of the respective coils 138 can display the shape image of the insertion portion 32 on the monitor 20. The user of the endoscope 12 can therefore visually recognize the position and posture of the insertion portion 32.

The detector 16 using this UPD device allows to always obtain the shape of the insertion portion 32 at the time of use of the endoscope 12. That is, when the user moves the insertion portion 32, the detector 16 updates the position/posture information, and the monitor 20 can display the shape after the movement.

Note that since the detector 16 and the video processor 14 are connected to each other, the monitor 18 connected to the video processor 14 can also display the position and posture of the insertion portion 32 of the endoscope 12 and can also display the updated position and posture without any time lag.

The driving face calculation unit 144 calculates driving faces of the bending portion 44 (the faces which are formed when the bending portion 44 bends) F1′ and F2′ based on the position/posture information of the distal end hard portion 42 out of the position/posture information of the insertion portion 32 (see FIG. 4A). In other words, the driving face calculation unit 144 calculates the positions and postures of the driving faces F1 and F2 as information of the driving faces F1′ and F2′. That is, the driving face calculation unit 144 can automatically obtain the driving face F1′ bending in the U and D directions and the driving face F2′ bending in the R and L directions by obtaining the position and posture of the bending portion 44. Note that the driving face F1′ is identical to the driving face F1 obtained from the observation optical system 74, and the driving face F2′ is identical to the driving face F2 obtained from the observation optical system 74.

An insertion support changeover switch (mode changeover switch) 150 is disposed near the angle knobs 62 and 64 of the operation portion 34 of the endoscope 12. The insertion support changeover switch 150 is used to switch between a support mode for supporting the insertion of the insertion portion 32 to the far side of the tubular body T and a normal mode. When, for example, the user keeps pressing the insertion support changeover switch 150 in the normal mode, the normal mode switches to the support mode. When, for example, the user cancels the pressed state of the switch 150, the support mode switches to the normal mode.

Note that the insertion support changeover switch 150 is preferably located at a position where the user operates the switch with his/her left index finger.

The endoscopic system 10 according to this embodiment operates in the following manner. The following will exemplify a case in which the bending portion 44 bends in the U and D directions.

The user of the endoscope 12 holds the operation portion 34 with his/her left hand, and holds the insertion portion 32 with his/her right hand, and then inserts the distal end hard portion 42 of the distal end of the insertion portion 32 from one end (anus) of the tubular body (e.g., the large intestine) T toward the far side (the other end). The user of the endoscope 12 moves the distal end hard portion 42 of the insertion portion 32 toward the far side of the tubular body T while grasping the internal state of the tubular body T on the monitor 18. When, for example, the distal end hard portion 42 approaches a crooked region of the tubular body T, for example, the sigmoid colon of the large intestine, the user cannot sometimes observe the far side of the tubular body T on the monitor 18.

Pressing the insertion support changeover switch 150 of the operation portion 34 will switch the normal mode to the support mode (step S1).

At this time, as shown in FIG. 4A, the driving face calculation unit 112 in the video processor 14 calculates the driving face F1(, F2) of the bending portion 44 (step S2). As shown in FIG. 4B, the peripheral information calculation unit 114 measures the distances between the wall surface of the tubular body T and the image sensors of the imaging units 86a and 86b on the driving face F1 calculated by the driving face calculation unit 112 at proper intervals (which can be set in advance by the operation panel 134) (step S3).

That is, the observation optical system 74 performs stereo imaging to obtain not only an image of the inner wall surface of the tubular body T but also the distances from the imaging units 86a and 86b arranged in the distal end hard portion 42 to the inner wall surface of the tubular body T on the image by using the principle of triangulation.

Assume that in this case, the peripheral information calculation unit 114 acquires distance information at the positions of points a, b, j, k on the driving face F1 of the observation image displayed on the monitor 18 in FIG. 4B based on the information of the images captured by the imaging units 86a and 86b. FIG. 4C shows distance information at the positions of the points a, b, j, k in FIG. 4B. That is, the apparatus converts the distance information obtained at the positions shown in FIG. 4B into the longitudinal section of the tubular body T shown in FIG. 4C.

As shown in FIG. 4C, it is possible to obtain a schematic shape (estimated sectional shape) of a longitudinal section of the tubular body T on the driving face F1 in the possible observation range by the observation optical system 74 (step S4).

Using the points a, b, j, k in FIG. 4C allows to recognize the schematic sectional shape of the tubular body T on the driving face F1. The peripheral information calculation unit 114 can then calculate an estimated wall surface of the tubular body T by using the point a, b, j, k.

It is easily understood that the larger the number of points where distance information is obtained, for example, the points a, b, j, k in FIGS. 4B and 4C, the higher the accuracy of an estimated wall surface, and vice versa.

The insertion path calculation unit 118 takes, for example, midpoints in the vertical direction from the near side in the section shown in FIG. 4C to the far side by using the calculated estimated wall surface. Connecting the respective midpoints from the near side to the far side will obtain the insertion path IP (step S5). The insertion path IP in FIG. 4C may be superimposed and displayed on the observation image shown in FIG. 4B.

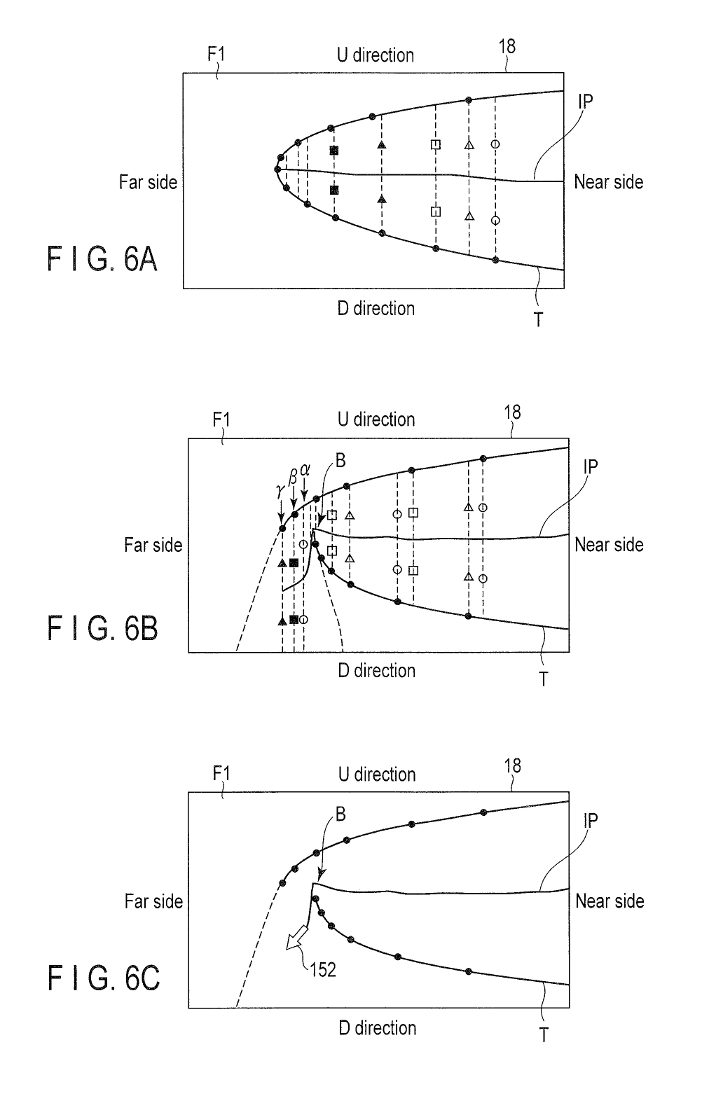

For example, as shown in FIG. 6A, a closed state on the far side may be obtained upon measurement of distances from the near side of the tubular body T to the far side on the driving face F1. This state indicates that even if the bending portion 44 bends within the driving face F1, i.e., the upward direction (U direction) or the downward direction (D direction), the insertion path IP does not exist on the far side. That is, as described above, when the midpoints on the estimated wall surface are taken and connected to each other to obtain the insertion path IP, although the insertion path IP can be calculated from the near side to a midway position, the insertion path IP does not extend through the far side.

In this case, the insertion path calculation unit 118 can determine that the insertion path comes to a dead end to be described below (step S5).

As shown in FIG. 6A, when the insertion path calculation unit 118 takes midpoints on the estimated wall surface on the driving face F1 and connects them to each other, the distant portion of the insertion path IP abuts against the estimated wall surface. In addition, the insertion path calculation unit 118 calculates sequentially calculates the gradient of the insertion path IP by differential operation or the like from the near side to the far side. In this case, if the gradient does not exceed a preset threshold, the insertion path calculation unit 118 can determine that a longitudinal section of the driving face F1 is closed on the far side.

In this case, it is possible to determine that there is an. insertion path on a driving face (e.g., the driving face F2) deviating from the current driving face F1. For this reason, the insertion portion 32 is made to pivot about its axis through, for example, 90° (either clockwise or counterclockwise). This pivoting operation will define new U and D directions and a new driving face F1. An insertion path ought to exist on the new driving face F1. Note that when the insertion portion 32 is made to pivot about its axis, since the insertion path IP may be detected when the insertion portion 32 is tilted to the far side by, for example, about 10°, making the insertion portion 32 pivot through 90° is merely an example.

FIG. 6B shows a case in which when midpoints on the estimated wall surface are taken and connected to each other, the insertion path IP has a portion (crooked region) which abruptly changes its direction, as indicated by reference symbol B. The insertion path calculation unit 118 sequentially calculates the gradient of the path at this time by differential operation or the like from the near side to the far side. When the calculated gradient exceeds a preset threshold, it is possible to determine that the corresponding portion is a crooked region B to which the distal end hard portion 42 of the insertion portion 32 should be guided. This allows the peripheral information detection unit 114, i.e., the peripheral information detection unit, to detect the crooked region B of the tubular body T, which exists on the driving face F1, as peripheral information.

Note that there is no wall surface of the tubular body T which is close to the points indicated by symbols α, β, and γ in the D direction in FIG. 6B. In this case, the insertion path calculation unit 118 calculates midpoints assuming that the lowermost end displayed on the monitor 18 is a wall surface.

That is, in the case shown in FIG. 6B, the insertion path calculation unit 118 can determine that there is the insertion path IP along which the distal end hard portion 42 of the insertion portion 32 can be moved to the far side.

In this manner, the insertion path calculation unit 118 can calculate the insertion path IP for the distal end hard portion 42 of the insertion portion 32 which extends from the near side in the tubular body T to the far side, and can automatically determine whether the distant portion of the driving face F1 observed by the observation optical system 74 is closed.

As shown in FIG. 6C, adding an arrow denoted by reference numeral 152 to an end portion of the insertion path IP can clearly present the user of the endoscope 12 the insertion path IP extending from the near side to the far side. Note that FIG. 6C simplifies the illustration of FIG. 6B, with the arrow 152 being only added to the distant end of the insertion path IP.

In the case shown in FIGS. 6B and 6C, the user of the endoscope 12 inserts the distal end hard portion 42 of the insertion portion 32 along the insertion path IP from the near side in the tubular body T to the far side. The user of the endoscope 12 then bends the bending portion 44 in the D direction by about 90° so as to see the far side of the crooked region B and hooks the bending portion 44 on the crooked region B. The user then pushes the insertion portion 32 to the far side while hooking the bending portion 44 on the crooked region B, and reduces the bending angle of the bending portion 44. This makes it possible to move the distal end hard portion 42 of the insertion portion 32 toward the far side of the crooked region B.

On the other hand, the detector 16 can always obtain the position and posture of the distal end hard portion 42 of the insertion portion 32, i.e., position/posture information, by using the shape calculation unit 142 (step S11). By using the position and posture calculated by the shape calculation unit 142, the driving face calculation unit 144 allows to obtain the driving faces F1′ and F2′ of the bending portion 44 (step S12).

The positional relationship calculation unit 116 in the video processor 14 matches the coordinate system of the driving face F1 calculated by the driving face calculation unit 112 of the video processor 14 with that of the driving face F1′ calculated by the driving face calculation unit 144 of the detector 16. In this case, the positional relationship between the image sensors of the imaging units 86a and 86b and the distal end face of the distal end hard portion 42 is known in advance, and the diameter of the distal end face of the distal end hard portion 42 is known in advance. For this reason, as shown in FIG. 4C, the positional relationship calculation unit 116 can calculate a positional relationship by superimposing the position of the distal end face of the distal end hard portion 42 of the insertion portion 32 or a schematic shape of the distal end hard portion 42 of the insertion portion 32 on an estimated sectional shape of the tubular body T including the crooked region B obtained by distance information. The monitor (presenting unit) 18 can display the positional relationship (step S20). The output unit (presenting unit) 106 outputs (presents) the positional relationship to an external device.

Note that it is possible to know the distances from the image sensors of the imaging units 86a and 86b of the insertion portion 32 to the inner wall of the tubular body T and display the insertion path IP for the insertion portion 32. This can output, onto the monitor 18, an instruction to, for example, push the insertion portion 32 straight from the near side in the tubular body T to the far side and then bend the insertion portion 32 in the U direction.

As described above, this embodiment can obtain the following effects.

Only operating the switch 150 of the operation portion 34 while performing observation using the observation optical system 74 can specify a direction (insertion path) in which the tube path of the tubular body T extends relative to the current position of the distal end hard portion 42 of the insertion portion 32. That is, it is possible to easily recognize the direction of the tubular body T as an observation target. If, for example, no insertion path exists on the driving face F1, it is possible to specify an insertion path on a new driving face F1 by operating the switch 150 of the operation portion 34 while making the insertion portion 32 pivot about its axis through, for example, 90°. This makes it possible to easily recognize an inserting direction when inserting the insertion portion 32 into the moving tubular body T such as the large intestine.

This embodiment can therefore provide the endoscopic system 10 which can support the insertion of the insertion portion 32 by allowing to grasp the direction in which the insertion portion 32 is to be moved, i.e., the insertion path IP, when inserting the insertion portion 32 of the endoscope 12 into the freely moving tubular body T such as the large intestine.

In addition, using the two imaging units 86a and 86b of the observation optical system 74 makes it possible to calculate the insertion path IP along which the distal end hard portion 42 of the insertion portion moves from the near side in the tubular body T to the far side by only measuring the distances between the image sensors in the distal end hard portion 42 of the insertion portion 32 and the wall surface on the driving face F1 in the U and D directions of the bending portion 44 in the tubular body T. This can minimize the number of devices used to calculate the insertion path IP. That is, in case that the endoscopic system 10 need not use any information obtained by superimposing the position and shape of the distal end hard portion 42 of the insertion portion 32 on a longitudinal section of part of the interior of the tubular body T and the endoscopic system 10 presents only the insertion path IP, this may eliminate the necessity to use the detector 16 capable of measuring the position and shape of the insertion portion 32 of the endoscope 12.

In addition, this embodiment can superimpose and display, on the monitor 18, the position of the distal end face of the distal end hard portion 42 of the insertion portion 32 or a schematic shape of the distal end hard portion 42 of the insertion portion 32 on a sectional shape of the interior of the tubular body T including the crooked region B, and can also output (present) the positional relationship to an external device. This makes it possible to easily recognize the moving amount and direction of the insertion portion 32 of the endoscope 12 from the near side in the tubular body T to the far side.

As shown in FIG. 6C, the arrow 152 is added to the distant portion of the insertion path IP, and hence it is easy for the user of the endoscope 12 to grasp the insertion path IP along which the distal end hard portion 42 of the insertion portion 32 is to be moved. It is possible to output (present) the insertion path IP to an external device.

Note that the insertion path calculation unit 118 can use various calculation methods other than the above calculation method as long as they allow to determine the insertion path (inserting direction) IP.

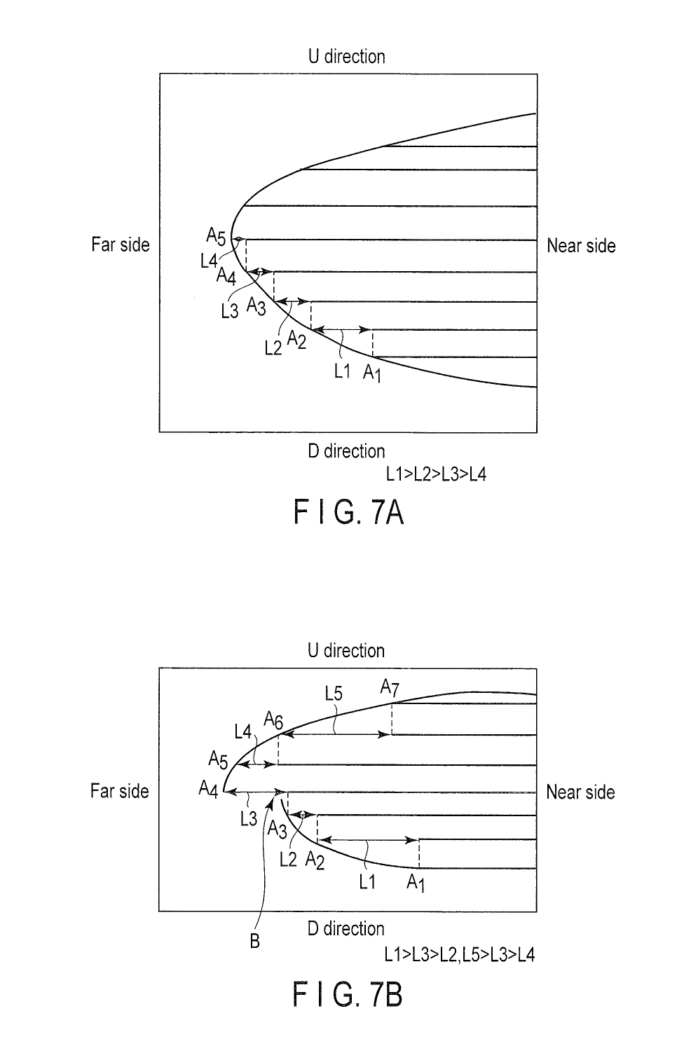

For example, the insertion path calculation unit 118 calculates differences L1, L2, L3, and L4 between the distances from the near side (near portion) to the far side (distant portion) at adjacent points A1, A2, A3, A4, and A5 in FIG. 7A. At this time, L1>L2 >L3>L4 holds. That is, the distance differences at the adjacent points A1, A2, A3, A4, and AS gradually decrease from the near side to the far side. If this state holds at all the points from the near side to the far side, the insertion path calculation unit 118 can determine that a region on the far side of a longitudinal section on the driving face F1 is closed.

In contrast to this, as shown in FIG. 7B, the insertion path calculation unit 118 calculates distance differences L1, L2, L3, L4, and L5 at adjacent points A1, A2, A3, A4, A5, A6, and A7. At this time, L1>L3>L2 and L5>L3>L4 hold. That is, the distance differences at the adjacent points A1, A2, A3, A4, A5, A6, and A7 gradually decrease from the near side (near portion) to the far side (distant portion). However, this state does not partly hold. In this case, the insertion path calculation unit 118 can determine that the crooked region B is formed in the region on the far side of the longitudinal section on the driving face F1.

Note that increasing the intervals between the adjacent points A1, A2, . . . , An will decrease the accuracy in calculating the insertion path IP, and decreasing the intervals can increase the accuracy.

In addition, the insertion path calculation unit 118 may use the following calculation methods.

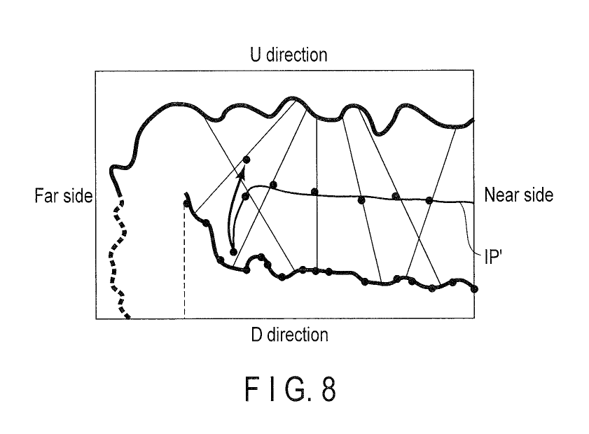

On the driving face F1, perpendicular lines are drawn from line segments connecting adjacent points on a section on the side in the D direction of the tubular body T in FIG. 8 toward the section on the side in the U direction of the tubular body T in FIG. 8. Plotting the midpoints of the extending perpendicular lines will obtain the locus denoted by reference symbol IP′ in FIG. 8. In this case, performing differential operation of the gradients of the line segments connecting the adjacent midpoints can obtain the magnitude of the amount of change in gradient. Deciding in advance a threshold for the amount of change in gradient allows to determine that the crooked region B is formed at a distant portion, if the amount of change in gradient is larger than the threshold, and to determine that the distant portion is closed, if the amount of change in gradient is small.

In addition, the insertion path calculation unit 118 may automatically determine the existence of the crooked region B by determining the bright portion/dark portion generated when irradiating an object with light emerging from the distal end face of the distal end hard portion 42 of the insertion portion 32 by using the illumination optical system 72 in addition to the observation optical system 74.

The insertion path IP calculation method to be used by the insertion path calculation unit 118 is not limited to only the above method. It is also preferable to use a combination of a plurality of calculation methods to improve the determination accuracy.

This embodiment has exemplified the case of using the stereo imaging scheme using the observation optical system 74 including the two objective lenses and the two imaging units 86a and 86b. However, it is also preferable to use a known distance image CMOS sensor or the like having a structure having only one imaging unit and capable of measuring an image and a distance.

Laser light can measure the distances between the imaging units (image sensors) and the inner wall of the tubular body T. It is possible to measure the distances between the distal end face of the distal end hard portion 42 of the insertion portion 32 and the inner wall surface of the tubular body T by scanning laser light on the driving face F1. In this case, a distance measuring device using laser light may be inserted in a treatment tool insertion channel or a distance measuring device incorporated in the insertion portion 32 may be used.

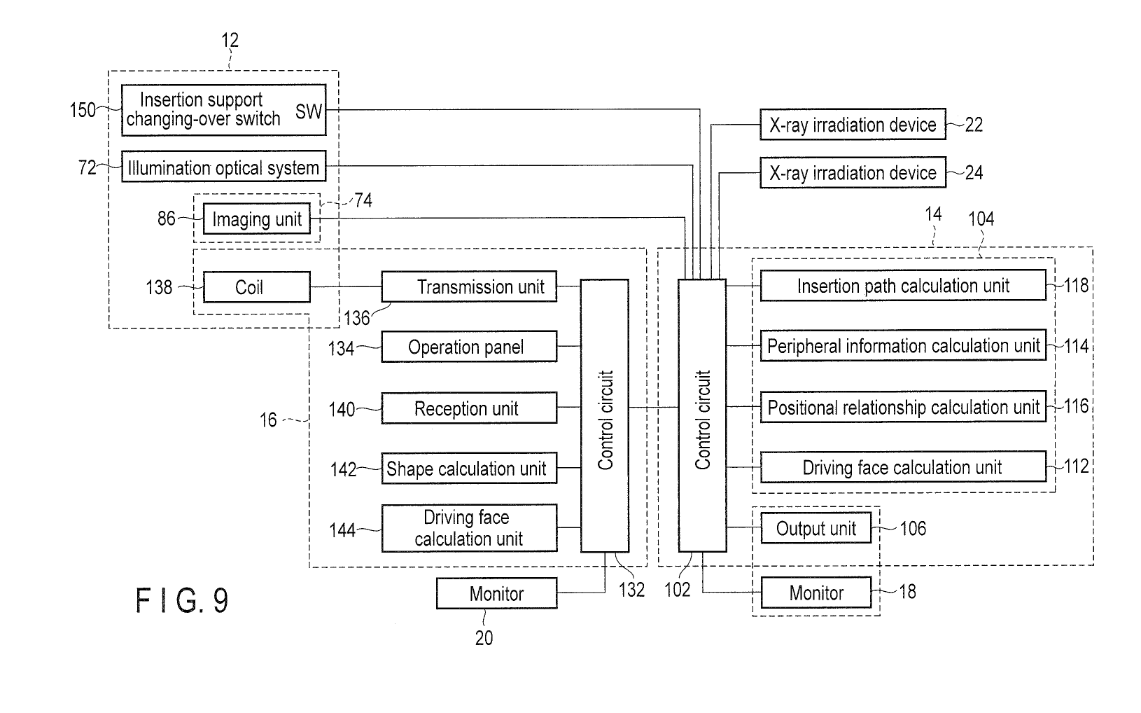

This embodiment has exemplified the case of defining the driving face F2 as well as the driving face F1. That is, the embodiment has exemplified the case of the bending portion 44 which bends in the four directions. However, for example, the bending portion 44 may have a structure which bends in only the two directions, i.e., the U and D directions. The second embodiment will be described next with reference to FIGS. 9 to 11. This embodiment is a modification of the first embodiment. The same reference numerals denote the same parts or parts having the same functions as in the first embodiment, and a detailed description of them will be omitted.

As shown in FIG. 9, an endoscopic system 10 according to this embodiment includes an endoscope 12, a video processor 14, a detector (position/posture detection unit) 16, monitors (presentation units) 18 and 20, and X-ray irradiation units (peripheral information detection units) 22 and 24. Although this embodiment will exemplify the use of the two X-ray irradiation units 22 and 24. However, the embodiment may use only one X-ray irradiation unit.

In addition, this embodiment will exemplify a case in which an observation optical system 74 includes one objective lens (not shown) and one imaging unit 86.

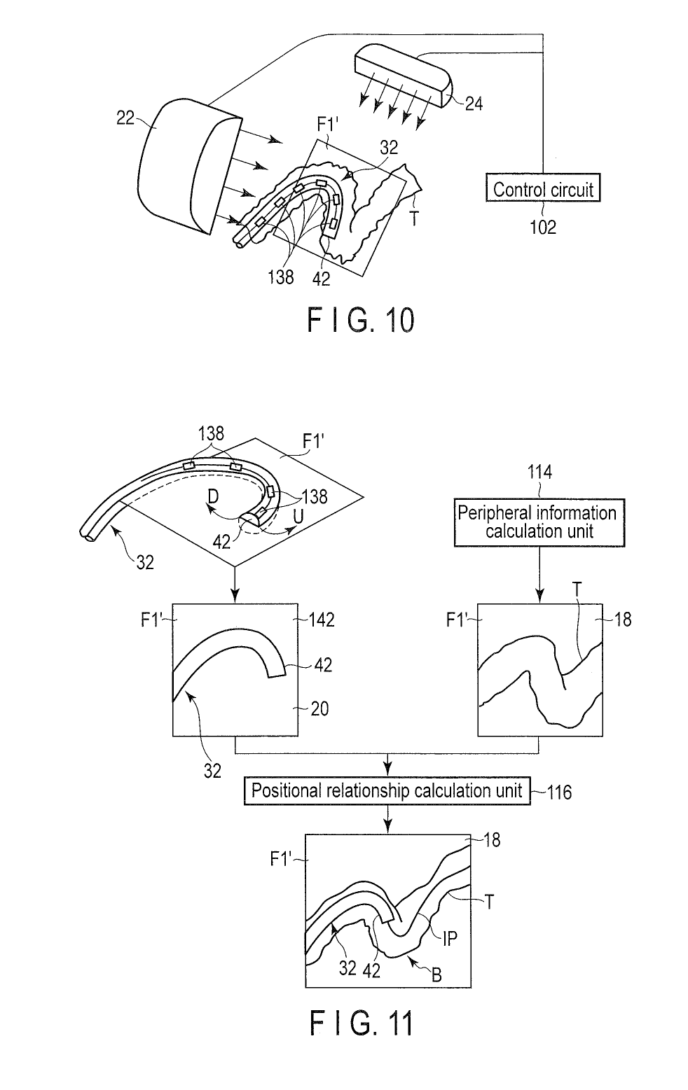

As shown in FIG. 10, the X-ray irradiation units 22 and 24 can emit X-rays from orthogonal positions and obtain X-ray tomographic images, respectively, while a distal end hard portion 42 of an insertion portion 32 of the endoscope 12 is inserted in a tubular body T.

The X-ray irradiation units 22 and 24 know, for example, coordinates concerning a bed 8 (see FIG. 1). It is therefore possible to use one X-ray irradiation device 22 so as to obtain an image of a driving face F1′ calculated by the detector 16 which knows coordinates concerning the bed 8 and to use the other X-ray irradiation device 24 so as to obtain an image of a driving face F2′ calculated by the detector 16 in the same manner.

Note that the X-ray irradiation units 22 and 24 and peripheral information calculation unit 114 acquire not only driving faces F1 and F2 but also peripheral X-ray tomographic images including the driving faces F1 and F2, and hence constitute a peripheral information detection unit. That is, the X-ray irradiation units 22 and 24 and the peripheral information calculation unit 114 can detect, as peripheral information, a crooked region B of the tubular body T existing on the driving faces F1 and F2.

As shown in FIG. 11, the peripheral information calculation unit (image processing unit) 114 performs image processing such as binarization processing for X-ray tomographic images (projection images) at this time to obtain sections of the tubular body T on the driving faces F1′ and F2′. The size of the tubular body T is known in advance by the X-ray irradiation units 22 and 24. In addition, the coordinates of the driving faces F1′ and F2′ are known by the detector 16, and the positions of the images obtained by applying X-rays from the X-ray irradiation units 22 and 24 are also known.

The positional relationship calculation unit 116 can therefore superimpose the projection images obtained by the X-ray irradiation units 22 and 24 on the driving face F1′ on the distal end hard portion 42 of the insertion portion 32 of the endoscope 12 of the detector 16, detected by the detector 16, by adjusting the size of the tubular body T of the X-ray tomographic image relative to the diameter of the distal end hard portion 42 of the insertion portion 32 or adjusting the diameter of the distal end hard portion 42 of the insertion portion 32 relative to the size of the tubular body T of the X-ray tomographic image. That is, the monitor 18 superimposes and displays the tubular body T and the distal end hard portion 42 of the insertion portion 32 of the endoscope 12. At this time, as the projection images obtained by the X-ray irradiation units 22 and 24, images depicting the portion from the near side where the distal end hard portion 42 of the insertion portion 32 exists to the far side can be acquired. As described in the first embodiment, it is therefore possible to display the midpoints between the edge portions of the tubular body T as an insertion path IP.

Note that the observation optical system 74 may be configured to include two objective lenses and two imaging units 86a and 86b so as to be capable of performing stereo imaging. In this case, the observation optical system 74 can extract the insertion path IP by obtaining X-ray tomographic images as well as being capable of performing the stereo imaging scheme described in the first embodiment. This makes it possible to improve the accuracy of the insertion path IP.

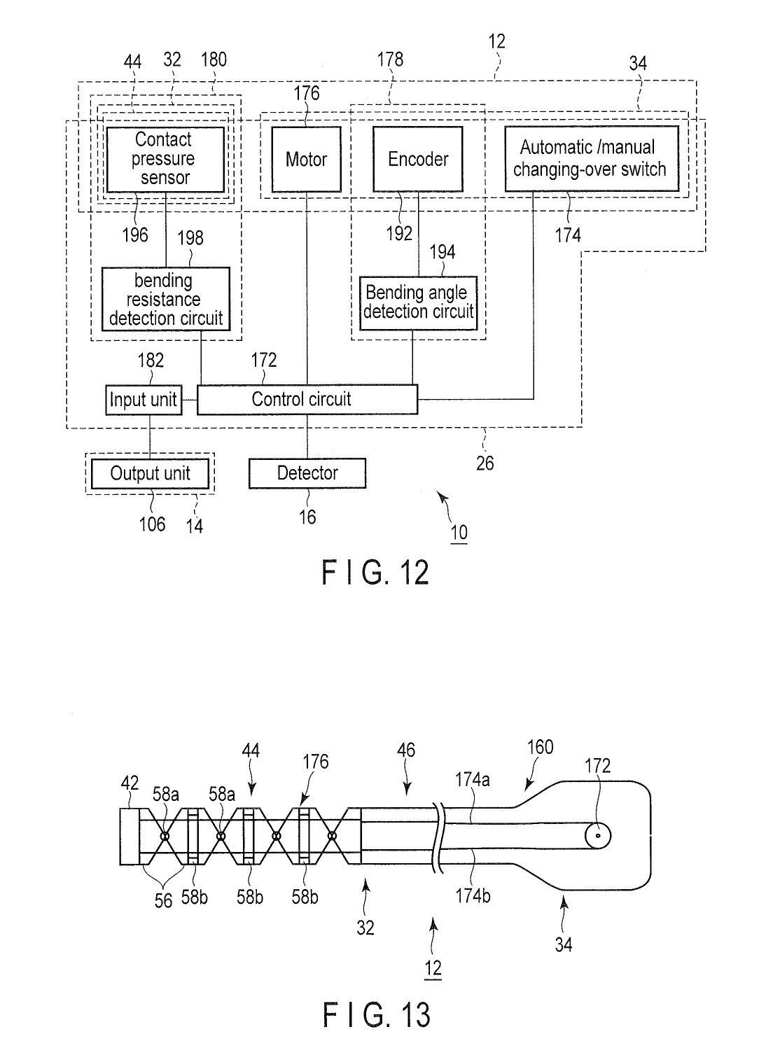

The third embodiment will be described next with reference to FIGS. 12 and 13. This embodiment is a modification of the first and second embodiments. The same reference numerals denote the same parts as those described in the first and second embodiments, and a detailed description of them will be omitted.

As shown in FIG. 12, an endoscopic system (insertion support apparatus for the insertion portion of an endoscope) 10 according to this embodiment includes an endoscope 12, a video processor 14, a detector (position/posture detection unit) 16, monitors (presentation units) 18 and 20, and an automatic bending driving device (automatic bending driving mechanism) 26.

This embodiment will exemplify the case of automatically performing bending operation in the U and D directions. However, the embodiment may automatically perform bending operation in the R and L directions as well as the U and D directions.

As shown in FIG. 13, a bending driving mechanism 160 of the endoscope 12 includes a pulley 162 disposed in the operation portion 34, angle wires 164a and 164b wound around the pulley 162, and a bending tube 166. The pulley 162 is coupled to the angle knobs 62 and 64 (see FIG. 1) disposed outside the operation portion 34. When the user operates the angle knobs 62 and 64 in the U direction, the angle wires 164a and 164b move in the axial direction via the pulley 162, and the bending tube 166 bends in the U direction. When the operator operates the angle knobs in the D direction, the bending tube 166 bends in the D direction.

As shown in FIG. 12, the automatic bending driving device 26 includes a control circuit 172, an automatic bending/manual bending changeover switch 174, a motor 176, a bending angle calculation unit 178, a bending resistance detection unit 180, and an input unit (connector) 182. Note that the input unit 182 inputs a signal from the output unit 106 of the video processor 14 described in the first embodiment to the control circuit 172.

The automatic bending/manual bending changeover switch 174 is provided, for example, near the angle knobs 62 and 64 (see FIG. 1) of the operation portion 34 to allow switching between an automatic bending mode of capable of bending the bending portion 44 in a predetermined case (when the insertion support changeover switch 150 is pressed) and a manual bending mode of manually bending the bending portion 44 even while the insertion support changeover switch 150 is pressed before the insertion of the insertion portion 32 into the tubular body T or during actual insertion of the insertion portion 32 into the tubular body T.

Note that the automatic bending/manual bending changeover switch 174 is preferably disposed near the insertion support changeover switch 150. For example, the operator can operate the automatic bending/manual bending changeover switch 174 with his/her left middle finger while operating the insertion support changeover switch 150 with his/her left index finger.

The motor 176 is connected to the pulley 162 in the operation portion 34. Therefore, rotating the driving shaft of the motor 176 will rotate the pulley 162.

The bending angle calculation unit 178 includes an encoder 192 which measures the rotation amount of the driving shaft of the motor 176 and a bending angle detection circuit 194 connected to the encoder 192.

The bending resistance detection unit 180 includes a contact pressure sensor 196 and a bending resistance detection circuit 198. The contact pressure sensor 196 is provided on the bending portion 44. Although not shown, a signal line connected to the contact pressure sensor 196 is connected to the bending resistance detection circuit 198 via the insertion portion 32 and the operation portion 34.

Note that the detector 16 can always detect the moving amount of the distal end hard portion 42 of the insertion portion 32.

For example, the user inserts the distal end hard portion 42 of the insertion portion 32 into the tubular body T from the near side of the tubular body T to the far side while the switch 174 of the automatic bending driving device 26 is switched to the automatic mode.

When the user presses the insertion support changeover switch 150 while the distal end hard portion 42 of the insertion portion 32 is placed in the tubular body T, the apparatus calculates the insertion path IP in the above manner. At this time, the insertion path IP is displayed on the monitor 18 and is output from the output unit 106. An output signal from the output unit 106 is input to the control circuit 172 of the automatic bending driving device 26.

If it is determined that the insertion path IP does not exist on the far side of the tubular body T (is closed), the output unit 106 outputs a signal for maintaining the shape of the bending portion 44 to the automatic bending driving device 26.

If it is determined that the insertion path IP exists on the far side of the tubular body T, the output unit 106 transfers a signal to the automatic bending driving device 26.

At this time, the automatic bending driving device 26 is synchronized with the detector 16. When the user moves the insertion portion 32 forward along the insertion path IP, the detector 16 automatically recognizes the moving amount of the insertion portion 32 in the axial direction. When the user moves the insertion portion 32 along the insertion path IP, the automatic bending driving device 26 bends the bending portion 44 so as to move the distal end face of the distal end hard portion 42 along the insertion path IP. This allows to hook the bending portion 44 on the crooked region B of the tubular body T. That is, it is possible to place the distal end face of the distal end hard portion 42 on the far side of the crooked region B.

Note that if the insertion portion 32 deviates from the insertion path IP and the bending portion 44 is in contact with the inner wall surface of the tubular body T, the contact pressure sensor 196 disposed on the bending portion 44 and the bending resistance detection circuit 198 detect the state. That is, the bending resistance detection unit 180 can detect from which position on the outer surface of the bending portion 44 a pressure is received. The motor 176 is then controlled to automatically adjust the bending angle of the bending portion 44 so as to reduce the contact pressure between the bending portion 44 and the inner wall surface of the tubular body T.

As has been described above, incorporating the automatic bending driving device 26 in the endoscopic system 10 makes it possible to automatically move the distal end hard portion 42 of the insertion portion 32 to the far side of the tubular body T. When guiding the distal end hard portion 42 of the insertion portion 32 from the near side of the crooked region B to the far side, the user of the endoscope 12 can save the labor of operating the endoscope 12.

Although the above embodiment has exemplified the case in which the insertion portion 32 has one bending portion 44, the insertion portion 32 preferably has two bending portions.

Although the endoscopic system 10 according to the above embodiments has been described as a medical system mainly applied to the large intestine, this system can be used for various applications such as industrial uses as well as medical uses.

### (Appendix)

An endoscopic system is characterized by including: an elongated insertion portion which is configured to be inserted into a tubular body and which includes, at a distal end portion, a bending portion configured to freely bend; a position/posture detection unit which is configured to detect a position and posture of the distal end portion as position/posture information; an operation position/posture calculation unit which is configured to calculate, as driving face information, a position and posture of a driving face on which the bending portion bends, based on the position/posture information; a peripheral information detection unit which is configured to detect a crooked region of the tubular body existing on the driving face as peripheral information based on the driving face information; a positional relationship calculation unit which is configured to calculate a positional relationship between the bending portion and the crooked region as positional relationship information based on the position/posture information, the driving face information and the peripheral information; and a presentation unit which is configured to present the positional relationship based on the position/posture information.

As described above, the position/posture detection unit can detect the position and posture of the distal end portion of the insertion portion, and the peripheral information detection unit can detect the crooked region of the tubular body on the driving face as peripheral information. The positional relationship calculation unit can calculate the positional relationship between the crooked region and the distal end portion of the insertion portion, and the presentation unit can present the positional relationship. Since the crooked region can be calculated by the peripheral information detection unit and presented together with the position/posture information of the distal end portion of the insertion portion, it is possible to present the direction in which the distal end portion of the insertion is to be moved, i.e., the insertion path. This can support the insertion of the insertion portion from the near side in the tubular body to the far side.

That is, it is possible to provide an endoscopic system which allows to grasp the direction in which the insertion portion is to be moved, i.e., the insertion path, when inserting the insertion portion of the endoscope into a freely moving tubular body such as the large intestine, thereby supporting the insertion of the insertion portion.

In addition, the peripheral information detection unit preferably includes: an X-ray tomographic image acquisition unit configured to acquire a shape of the tubular body along the driving face calculated by the position/posture detection unit, and an image processing unit configured to extract an outline of the tubular body including a portion from a near side in the tubular body, in which the distal end portion of the insertion portion is placed, to a far side in the tubular body based on an X-ray tomographic image acquired by the X-ray tomographic image acquisition unit.

The peripheral information detection unit can therefore acquire an X-ray tomographic image including a longitudinal section (outline) of a tubular body and obtain a desired state, i.e., a longitudinal section on the driving face, by performing image processing for the X-ray tomographic image.

An endoscopic system is characterized by including: an insertion portion which includes a distal end portion and a bending portion whose driving face is defined by bending in at least two directions, and which is configured to be inserted into the tubular body; a distance measuring mechanism which is configured to acquire distance information in the driving face between an inner wall of the tubular body on a far side therein and the distal end portion of the insertion portion while the distal end portion of the insertion portion is placed on a near side in the tubular body; an insertion path calculation unit which is configured to calculate an insertion path for the distal end portion of the insertion portion which extends from the near side on which the distal end portion of the insertion portion is placed, to the far side, based on the distance information; and a presentation unit which is configured to present the insertion path for the distal end portion of the insertion portion which extends from the near side to the far side.

As described above, the distance measuring mechanism acquires the distances between the distal end portion of the insertion portion and the inner wall of the tubular body on the far side on the driving face, the insertion path calculation unit calculates an insertion path and they are presented on the presentation unit, thereby presenting the direction in which the distal end portion of the insertion portion is to be moved, i.e., the insertion path. This makes it possible to support the insertion of the insertion portion from the near side in the tubular body to the far side.

That is, it is possible to provide an endoscopic system which allows to grasp the direction in which the insertion portion is to be moved, i.e., the insertion path, when inserting the insertion portion of the endoscope into a freely moving tubular body such as the large intestine, thereby supporting the insertion of the insertion portion.

In addition, the distance measuring mechanism preferably includes an optical system which can acquire the distances between the inner wall of the tubular body on the far side therein and the distal end portion of the insertion portion on the driving face.

Incorporating an optical system in the insertion portion of the endoscope or inserting an optical system in a channel can easily measure the distances between the distal end portion of the insertion portion and the inner wall of the tubular body on the far side.

Furthermore, the endoscopic system preferably further includes: a position/posture detection unit which is configured to detect a position and posture of the distal end portion of the insertion portion in the tubular body as position/posture information and which is configured to calculate the driving face based on the position/posture information; a positional relationship calculation unit which is configured to calculate a positional relationship of the insertion path with respect to the distal end portion of the insertion portion based on the position/posture information and the distance information; and an automatic bending driving mechanism which is connected to the presentation unit and which is configured to automatically bend the bending portion toward the insertion path presented by the presentation unit.

This makes it possible to more easily insert the insertion portion to the far side of the tubular body while bending the bending portion along the insertion path presented by the presentation unit.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscopic system comprising:
an elongated insertion portion which is configured to be inserted into a tubular body and which includes, at a distal end portion, a bending portion configured to freely bend;
a position/posture detection unit which is configured to detect a position and posture of the distal end portion as position/posture information;
an operation position/posture calculation unit which is configured to calculate, as driving face information, a position and posture of a driving face on which the bending portion bends, based on the position/posture information;
a peripheral information detection unit which is configured to detect a crooked region of the tubular body existing on the driving face as peripheral information based on the driving face information;
a positional relationship calculation unit which is configured to calculate a positional relationship between the bending portion and the crooked region as positional relationship information based on the position/posture information, the driving face information and the peripheral information; and
a presentation unit which is configured to present the positional relationship based on the positional relationship information.

2. The endoscopic system according to claim 1, wherein the peripheral information detection unit includes an optical system configured to acquire a distance between an inner wall of the tubular body on a far side therein and the distal end portion of the insertion portion on the driving face.

3. The endoscopic system according to claim 1, wherein the peripheral information detection unit includes:
an X-ray tomographic image acquisition unit configured to acquire a shape of the tubular body along the driving face calculated by the position/posture detection unit, and
an image processing unit configured to extract an outline of the tubular body including a portion from a near side in the tubular body, in which the distal end portion of the insertion portion is placed, to a far side in the tubular body based on an X-ray tomographic image acquired by the X-ray tomographic image acquisition unit.

4. The endoscopic system according to claim 1, wherein the peripheral information detection unit is configured to detect a shape of the tubular body on the driving face, is configured to calculate an insertion path for the insertion portion based on the shape of the tubular body, and is configured to calculate the crooked region based on the insertion path.

5. The endoscopic system according to claim 4, further comprising a bending direction calculation unit configured to calculate a crooked direction of the crooked region based on the insertion path.

6. The endoscopic system according to claim 1, further comprising a window display unit connected to the presentation unit and configured to display the positional relationship presented by the presentation unit on a window.

7. The endoscopic system according to claim 6, wherein the window display unit is configured to display a crooked direction of the crooked region along the driving face.

8. The endoscopic system according to claim 1, further comprising an automatic bending driving mechanism connected to the presentation unit and configured to automatically bend the bending portion toward the crooked region based on the positional relationship presented by the presentation unit.

9. The endoscopic system according to claim 1, wherein
the bending portion includes a plurality of bending pieces and pivot shafts pivotally coupling the bending pieces to each other, and
the driving face is defined by the pivot shafts.

10. An endoscopic system comprising:
an insertion portion which includes a distal end portion and a bending portion whose driving face is defined by bending in at least two directions, and which is configured to be inserted into a tubular body;
a distance measuring mechanism which is configured to acquire distance information on the driving face between an inner wall of the tubular body on a far side therein and the distal end portion of the insertion portion while the distal end portion of the insertion portion is placed on a near side in the tubular body;
an insertion path calculation unit which is configured to calculate an insertion path for the distal end portion of the insertion portion which extends from the near side on which the distal end portion of the insertion portion is placed, to the far side, based on the distance information; and
a presentation unit which is configured to present the insertion path for the distal end portion of the insertion portion which extends from the near side to the far side.

11. The endoscopic system according to claim 10, wherein the distance measuring mechanism includes an optical system configured to acquire a distance between the inner wall of the tubular body on the far side therein and the distal end portion of the insertion portion on the driving face.

12. The endoscopic system according to claim 11, wherein the optical system includes an imaging unit disposed in the insertion portion.

13. The endoscopic system according to claim 10, further comprising:
a position/posture detection unit which is configured to detect a position and posture of the distal end portion of the insertion portion in the tubular body as position/posture information and which is configured to calculate the driving face based on the position/posture information;
a positional relationship calculation unit which is configured to calculate a positional relationship of the insertion path with respect to the distal end portion of the insertion portion based on the position/posture information and the distance information; and
an automatic bending driving mechanism which is connected to the presentation unit and which is configured to automatically bend the bending portion toward the insertion path presented by the presentation unit.

14. The endoscopic system according to claim 10, wherein
the bending portion includes a plurality of bending pieces and pivot shafts pivotally coupling the bending pieces to each other, and
the driving face is defined by the pivot shafts.
